(19) United States
(12) Patent Application Publication
(10) Pub. No.: US 2023/0405327 A1
(43) Pub. Date: Dec. 21, 2023

(54) VAGUS NERVE STIMULATION PUPILLOMETRY FOR ELECTRODE SELECTION AND TITRATION

(71) Applicant: The Alfred E. Mann Foundation for Scientific Research, Valencia, CA (US)

(72) Inventors: Brian Michael SHELTON, Altadena, CA (US); Brian MECH, Buffalo, MN (US); Valma KLEIN, Santa Clarita, CA (US); Neil TALBOT, La Crescenta, CA (US); Robert GREENBERG, Los Angeles, CA (US)

(21) Appl. No.: 18/180,591

(22) Filed: Mar. 8, 2023

Related U.S. Application Data
(60) Provisional application No. 63/350,748, filed on Jun. 9, 2022.

(51) Int. Cl. A61N 1/36; A61N 1/372

(52) U.S. Cl. CPC A61N 1/36139; A61N 1/37211; A61N 1/36053

(57) ABSTRACT

The present disclosure generally relates to systems and methods for a stimulation system. Various aspects of the present disclosure relate generally to the treatment of epilepsy, depression and/or other conditions in a subject using a nerve stimulator and, more particularly, to an effective and quicker titration method for selection of stimulation parameters of the nerve stimulator using biological markers that indicate potential therapeutic effects. Some aspects of the present disclosure may utilize a pupillometry sensor synchronized with the VNS stimulation system to compare pupil size of a subject with stimulation on and off and for obtaining filtered pupil response measurements after stimulating each electrode while modulating the stimulation parameters. Thus, aspects of the present disclosure allow for quicker titration of programmable stimulation parameters (e.g., stimulation pulse amplitude) to levels that cause a therapeutic result in a matter of minutes instead of months as compared to related VNS systems.

### CROSS-REFERENCE TO RELATED APPLICATION

This application claims the benefit of U.S. Provisional Application Ser. No. 63/350,748, entitled “VNS PUPILLOMETRY FOR ELECTRODE SELECTION AND TITRATION,” filed on Jun. 9, 2022, and is expressly incorporated by reference herein in its entirety.

### TECHNICAL FIELD

The present disclosure generally relates to systems and methods for treating epilepsy using a vagus nerve stimulation (VNS) and methods of treating medical conditions related thereto.

### BACKGROUND

Epilepsy and depression are two extremely common maladies. Both conditions can be treated under appropriate circumstances with VNS systems. VNS entails the surgical implantation of a device into a patient's chest area under the skin to stimulate the patient's vagus nerve with electrical impulses. The vagus nerve originates from the brainstem and traverses both sides of the neck down to the chest and abdomen. The VNS system sends electrical signals via the vagus nerve to the brain. A lead wire connects the device to the vagus nerve via a cuff with one or more electrodes inside the cuff. VNS has been shown to be helpful in many cases for reducing the number and severity of seizures, particularly for patients who are less responsive to more non-invasive methods such as oral medication. VNS has also been shown to reduce depression in certain treatment-resistant patients.

The goal of titration is to optimize output current to a therapeutic level that is well-tolerated by the patient while also limiting cardiac and other undesirable side effects for the patient. The titration speed can vary and depends on the comfort level of the healthcare provider and patient. Patient tolerability relates to the degree at which the patient feels sensations or experiences side effects associated with VNS such as hoarseness and voice alterations. Other side effects may include paresthesia, coughing, and shortness of breath. Cardiac side effects may include tachycardia, which typically refers to a fast resting heart rate of usually more than 100 beats per minute for most adults, and bradycardia, which refers to a slow heart rate that is typically less than beats per minute for most adults. Both tachycardia and bradycardia may be life threatening since episodes of tachycardia lasting more than a few seconds may be life-threatening or cause the heart to stop and bradycardia may be associated with an increased risk of sudden unexplained death. Accordingly, lower pulse widths and frequency settings may be programmed to manage stimulation associated side effects. Therefore, it is helpful for healthcare providers to conduct increases in output current at a rate that is tolerable and comfortable (or be less likely to be aroused from sleep) for the patient, but also quick enough that the patient begins to realize therapeutic benefits.

For example, LiveNova PLC has a VNS system that increases the stimulation current by a step size at a specific interval (e.g., increase by 0.1 mA weekly) programmed by a clinician. However, the problem with this approach is that the stimulation starts low and increases slowly. This means that it takes a long period of time until the VNS system is properly titrated. Accordingly, it would be helpful if clinicians had a biological marker that indicated the potential therapeutic effect of VNS systems such that the pulse amplitudes could be selected systematically and quickly instead of arbitrarily and slowly. Thus, monitoring biological markers that indicate the potential therapeutic effect would result in a quicker and more effective titration method for VNS systems.

### BRIEF SUMMARY OF EXEMPLARY ASPECTS OF THE DISCLOSURE

The systems and methods for vagus nerve stimulation (VNS) described herein address various needs in the art, e.g., by systematically selecting and determining appropriate stimulation parameters for VNS based on analyzing biological markers that indicate potential therapeutic effects. For instance, since pupil size increases as a result of noradrenalin release in response to afferent stimulation on the cervical vagus nerve (VN), a pupillometry sensor may monitor and/or track a magnitude of pupil size change that occurs when stimulation is delivered to the subject. In an embodiment, a pupillometry sensor is synced with the VNS system to obtain pupil measurements of the subject when stimulation is on and off. In practice, such systems can provide a quick and accurate means for titrating VNS, resulting in a more efficient and quicker titration for optimal therapy.

In a first general aspect, the disclosure provides a stimulation system, comprising: a nerve stimulator implanted in a subject and configured to deliver a stimulation therapy to the subject based on transmitting electrical stimulation pulses to a VN of the subject via one or more electrodes; and a pupillometry sensor associated with the subject and configured to measure a pupil size of the subject, where the nerve stimulator comprises a controller communicatively coupled to the nerve stimulator and the pupillometry sensor. The controller is configured to cause the nerve stimulator to deliver an electrical stimulation pulse to the VN via a first electrode among the one or more electrodes on the VN according to at least one stimulation parameter, obtain, via the pupillometry sensor, a change of measurement in pupil size of the subject according to the delivered electrical stimulation pulse to the VN via the first electrode, and control administration of the stimulation therapy based on the obtained change of measurement in pupil size.

In some aspects, the controller is further configured to determine whether the electrical stimulation pulse causes an action potential in afferent fibers of the subject based on the obtained change of measurement in pupil size, and based on a determination that the electrical stimulation pulse causes an action potential in afferent fibers of the subject, store, in memory, the at least one stimulation parameter as a minimum threshold parameter for the first electrode.

In some aspects, the determining is performed at an external device corresponding to a discrete controller of the stimulation system, a computer, a smart phone, or a tablet.

In some aspects, the determining is performed at an internal (e.g., implanted) device corresponding to a wirelessly connected discrete controller, a wire-connected discrete controller, or a controller integrated with or housed within the stimulation system.

In some aspects, the determining is performed remotely by cloud computing via data communicated wirelessly from either external or internal (e.g., implanted) parts of the stimulation system.

In some aspects, the at least one stimulation parameter includes at least one of a pulse amplitude a pulse width, a pulse frequency, ramp-on rate, ramp-off rate and/or duty cycle of the electrical stimulation pulses.

In some aspects, the stimulation system further comprises: an electromyography (EMG) sensor configured to obtain EMG measurements of the subject, wherein the controller is further configured to: obtain, via the EMG sensor, the EMG measurements of the subject according to the delivered electrical stimulation pulse to the VN simultaneously with obtaining the pupil measurements of the subject; and setting a maximum value of the at least one stimulation parameter based on the obtained EMG measurements of the subject.

In some aspects, the stimulation system further comprises one or more light sources coupled to the pupillometry sensor, wherein the controller is further configured to control the one or more light sources to obtain a nominal pupil size of the subject.

In some aspects, the stimulation system further comprises one or more light sources coupled to the pupillometry sensor, wherein the controller is further configured to maintain a constant level of light to the eye(s) of the subject being monitored for changes in pupil size.

In some aspects, obtaining pupil measurement of the subject according to the delivered stimulation to the VN further comprises: obtaining, via the pupillometry sensor, a first pupil size measurement when the nerve stimulator delivers an electrical stimulation pulse to the VN via the first electrode among the one or more electrodes according to the at least one stimulation parameter, obtaining, via the pupillometry sensor, a second pupil size measurement when the nerve stimulator ceases delivering electrical stimulation pulses to the VN via the first electrode among the one or more electrodes, and storing, in memory, the first pupil size measurements associated with the one or more parameters and the second pupil size measurement or a delta between the first pupil size measurement and the second pupil size measurement.

In some aspects, the first pupil size measurement and the second pupil size measurement of the subject are obtained in relation to a timing of a stimulation pulse train delivered by the nerve stimulator.

In some aspects, the controller is further configured to: determine whether the electrical stimulation pulse causes an action potential in afferent fibers of the subject based on the obtained change of measurement in pupil size, based on a determination that the electrical stimulation pulse does not cause an action potential in afferent fibers of the subject, cause the nerve stimulator to deliver an electrical stimulation pulse to the VN via a first electrode among the one or more electrodes on the VN according to an increase in the least one stimulation parameter, and obtain one or more subsequent measurements of change in pupil size of the subject according to the delivered electrical stimulation pulse to the VN via the first electrode.

In some aspects, the controller is further configured to: cause the nerve stimulator to deliver an electrical stimulation pulse to the VN via a second electrode among the one or more electrodes on the VN according to at least one stimulation parameter, wherein the second electrode is paired with the first electrode, obtain, via the pupillometry sensor, a change of measurement in pupil size of the subject according to the delivered stimulation to the VN via the second electrode, determine whether the electrical stimulation pulse causes an action potential in afferent fibers of the subject based on the obtained change of measurement in pupil size, and based on a determination that the electrical stimulation pulse causes an action potential in afferent fibers of the subject, storing, in memory, the at least one stimulation parameter as a minimum threshold for the first and the second electrode.

In some aspects, the stimulation system further comprises: a sensor configured to detect, measure, and/or monitor one or more biomarkers of the subject, wherein the controller is further configured to: receive a biomarker data for the subject, and use the biomarker data when determining therapeutic effects of VN stimulation.

In some aspects, wherein the one or more biomarkers comprises a heart rate variability (HVR) of the subject.

In some aspects, the controller is further configured to: administer, cease administering, titrate, or adjust a level of stimulation administered to the subject based on the one or more biomarkers.

In some aspects, the controller is further configured to: obtain a selection of an electrode from among the one or electrodes, and cause the nerve stimulator to deliver electrical stimulation pulse to the VN via the selected electrode according to the at least one stimulation parameter.

In some aspects, the controller is further configured to allow the subject and/or a remote operator to adjust the at least one stimulation parameter and allow the subject to provide feedback per a stimulation comfort level or discomfort of the subject.

In some aspects, controlling the administration of the stimulation therapy further comprises administering, ceasing administration, titrating or adjusting a level of electrical stimulation pulse administered to the subject.

In a second general aspect, the disclosure provides methods for VNS, comprising: providing a nerve stimulator implanted in a subject and configured to deliver a stimulation therapy to a subject based on transmitting electrical stimulation pulses to a vagus nerve (VN) of the subject via one or more electrodes, providing a pupillometry sensor associated with a subject and configured to measure a pupil size of the subject, delivering an electrical stimulation pulse to the VN via a first electrode among the one or more electrodes on the VN according to at least one stimulation parameter, obtaining, via the pupillometry sensor, a change of measurement in pupil size of the subject according to the delivered electrical stimulation pulse to the VN via the first electrode, and controlling administration of the stimulation therapy based on the obtained pupil size measurement.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1A is a diagram illustrating an exemplary embodiment of a VNS system for treating epilepsy using an implantable VNS stimulator coupled via a lead wire to drive a cuff electrode on the vagus nerve and a pupillometry sensor system to monitor pupil size.

FIG. 1B is a diagram illustrating an exemplary embodiment of a VNS system for treating epilepsy using an implantable VNS stimulator coupled via a lead wire to drive a cuff electrode on the vagus nerve and a pupillometry sensor system to monitor pupil size.

FIG. 2 is a diagram illustrating an exemplary embodiment of a VNS stimulator including a pulse generator and a controller configured to modulate one or more parameters of the electrical stimulation pulse and to synchronize the implantable VNS stimulation with the pupillometry sensor system.

FIG. 3 is a diagram illustrating an exemplary embodiment of a wire and electrode cuff for use in VNS stimulation.

FIG. 4 is a conceptual flow diagram of a process for stimulation therapy in a subject according to an exemplary embodiment.

FIG. 5 is a conceptual flow diagram of a process for stimulation therapy in a subject according to an exemplary embodiment.

FIG. 6 is a conceptual flow diagram of a process for stimulation therapy in a subject according to an exemplary embodiment.

FIG. 7 is a conceptual flow diagram of a process for stimulation therapy in a subject according to an exemplary embodiment.

FIG. 8 is a conceptual flow diagram of a process for stimulation therapy in a subject according to an exemplary embodiment.

FIG. 9 is a conceptual flow diagram of a process for stimulation therapy in a subject according to an exemplary embodiment.

FIG. 10 is a conceptual flow diagram of a process for stimulation therapy in a subject according to an exemplary embodiment.

FIG. 11 is a conceptual flow diagram of a process for stimulation therapy in a subject according to an exemplary embodiment.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Various aspects of the present disclosure relate generally to the treatment of epilepsy in a subject using a VNS stimulator and, more particularly, to an effective and quicker titration method for selection of stimulation parameters of the VNS stimulator using biological markers indicating potential therapeutic effects. Some aspects of the present disclosure may utilize a pupillometry sensor synchronized with the VNS stimulator to compare pupil size of a subject with stimulation on and off and for obtaining filtered pupil response measurements after stimulating each electrode while modulating the stimulation parameters.

Some aspects of the present disclosure more specifically relate to determining a minimum stimulation parameter setting to cause an action potential afferent fiber of the subject for each electrode of a multi-electrode stimulation cuff. This allow for a comparison of the efficacy of each electrode of a multi-electrode cervical VNS stimulation lead and, thus, allows for precise selection or deselection of one or more electrodes depending on therapeutic effects and side effects. For instance, it may be possible that the electrode or electrode pair that elicits the greatest response may also result in undesirable side effects such as causing comfort issues and/or cardiac side effects. In this case, information regarding the undesirable side effects may be collected and used to modify the algorithm that is employed to select the desirable electrode or electrode pair.

Thus, aspects of the present disclosure allow for quicker titration of programmable stimulation parameters (e.g., stimulation pulse amplitude) in a nerve stimulator to levels that cause a therapeutic result in a matter of minutes instead of months as compared to related VNS systems. This will help a clinician choose a selection of electrodes that have the greatest therapeutic outcome with the fewest side effect. Aspects of the present disclosure also allow for automatic titration of programmable stimulation parameters to the point where seizure occurrence is reduced with minimal stimulator use. This allows for longer device longevity and longer recharge intervals for rechargeable system. Some aspects of the present disclosure also allow for selection of electrodes (and deselection of electrodes) in a multi-electrode stimulation cuff in order to achieve the greatest therapeutic outcome.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

When activated, a VNS system delivers a stimulation pulse train with the pulse amplitude being the output current. In related VNS systems, a default pulse width and signal frequency are arbitrarily selected at an initial phase of treatment. Generally, the initial phase of treatment begins a few weeks after the VNS system has been implanted into the subject in order to allow the subject to heal from surgery. The pulse amplitude is then arbitrarily increased at regular intervals (e.g., on a weekly basis) until side effects are observed. The level of vagus nerve stimulation is dependent on a combination of different stimulation parameters. As an example, shorter pulse widths may require higher output currents to achieve a similar response.

Common titration strategies for VNS prioritize monitoring a subject's tolerability of side effects from stimulation by slow and small increases in stimulation intensity over several months. For example, the pulse widths and frequency settings may be initially programmed to be very low in order to manage stimulation associated side effects. Subject tolerability is partially based on how quickly the side effects can be perceived and/or reported by subjects and the delayed onset of clinical benefits from VNS. However, excessive caution during the titration phase may significantly delay target dosing or prevent a subject from reaching a therapeutic dose entirely. In addition, subject tolerability also considers limiting (or preventing) cardiac side effects such as tachycardia and bradycardia.

Additionally, a VNS system with multiple electrodes has a compounded complication of selecting electrodes that have the greatest therapeutic outcome with the fewest side effects. In multi-electrode systems it can take many months to determine the impact that a single electrode has on the reduction of seizure frequency. Therefore, it would be helpful to have a systematic and accurate method to select and de-select electrodes (e.g., cathodes or anodes) and/or pairs of electrodes that elicit the greatest pupillary response while also minimizing undesired side effects.

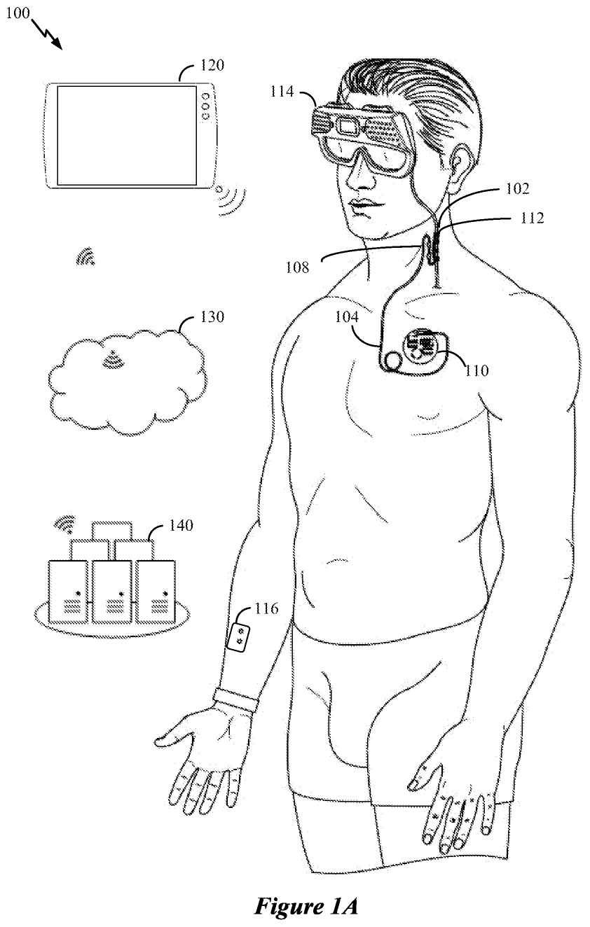

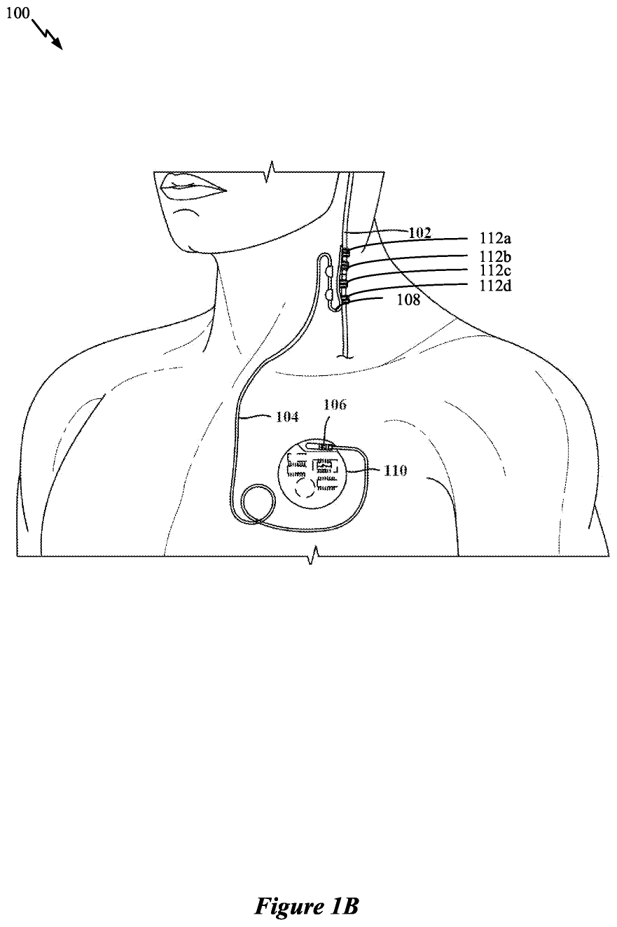

FIG. 1A is a diagram illustrating an exemplary embodiment of a VNS system for treating epilepsy using a VNS stimulator 110 implanted under the skin in the chest of a subject and coupled via a lead wire 104 to drive a cuff electrode 108 (or a plurality of electrodes inside the cuff) on the vagus nerve 102. As will be shown in greater detail in FIG. 1B, electrode pairs 112 at the end of a lead wire 104 may connect to the vagus nerve 102 by gently wrapping the electrodes around a left vagus nerve through an incision made in the neck of the subject. The stimulation of the vagus nerve 102 using electrical pulses is believed to stabilize abnormal electrical activity in the brain which can lead to seizures.

A VNS stimulator 110 may also include a rechargeable or primary cell, single-use battery for transmitting electrical pulses using a selectable pulse width and frequency. The VNS stimulator 110 may further include an outlet portion for enabling a connection between the VNS stimulator 110 and the lead wire 104, which enables current flow to the vagus nerve 102 via the cuff electrode 108 with electrode(s) 112, and/or a connection between VNS stimulator 110 and a pupillometry system 114.

In some aspects, the VNS system 100 may comprise a pupillometry system 114 configured to measure a spontaneous variation of the pupil diameter and the pupillary light reflex in the subject. Pupillometry is a non-invasive objective monitoring technique which is used to evaluate the autonomous nervous system and as an objective marker of light input from the retina. Since pupil size increases slightly as a result of noradrenalin released in response to afferent stimulation on the cervical vagus nerve, the magnitude of the pupil size change that occurs when stimulation is delivered to the vagus nerve 102 may be used as a biological marker to indicate a potential therapeutic effect of the stimulation. However, there are a few possible causes of pupil size changes that should be controlled or factored out. First, the ambient lighting may be controlled since lighting itself can cause pupil size change with a magnitude (e.g., multiple mm) that is much greater than caused by the stimulation (e.g., 1 mm). Second, side effects that cause discomfort may also have an effect on reducing pupil size. In this case, electromyography (EMG) measurements of side effects may be obtained and utilized to eliminate this variable. EMG is a medical test performed to evaluate and record the electrical activity (e.g., electromyogram) produced by skeletal muscles using EMG sensors 116. The EMG sensors 116 are configured to measure small electrical signals generated by a subject's muscles when the subject moves. This may include movements such as the subject lifting his arm, clenching his fist, or moving his finger. The combinatorial effect of these two factors may result in an even quicker titration to an optimal therapy.

As shown in FIG. 1A, the pupillometry system 114 may include a pupillometry sensor may be mounted onto a pair of glasses or into a pair of goggles in order to simplify the process of tracking the pupil and monitoring the pupil size of the subject. Since the pupillometry system 114 is head-mounted, the camera of the pupillometry system 114 will move with the subject's head. To account for ambient lighting, the glasses or goggles may be tinted and have side shields to control the ambient lighting of the pupil and consequently control the pupil's response to ambient light. However, since pupil response depends upon the pupil size, the tint or side shields may cause the subject's pupils to become too large. Therefore, a small light source (e.g., LED) may be added into the side of the googles or glasses to control the nominal pupil size for maximum pupil response.

The pupillometry system 114 may also be synchronized with the VNS system 100 such that pupil measurements are obtained based on the timing of stimulation delivery to the vagus nerve 102. This allows a comparison of the pupil size with stimulation on and off. In some aspects, during stimulation, it can take as long as 5 seconds to record any pupil response and it can take up to 10 seconds or longer to achieve a maximum amplitude. Recovery of the pupil diameter can also take as long as 40 seconds. In sum, it may take approximately a total of 45-60 seconds on average for each pupil measurement reading. Thus, the synchronization between the pupillometry system and the VNS system 100 may be accurate to within approximately 1 second.

In some aspects, an external device 120 may synchronize the pupillometry system 114 and the VNS system 100 by communicating with the VNS stimulator 110 to obtain the timing of the stimulation pulse trains. The external device 120 may then transmit this information to the pupillometry system 114 for synchronizing the pupillometry system 114 with the VNS stimulator 110 to obtain measurements with stimulation on and off. Specifically, the external device 120 may obtain pupil size measurement of the subject according to the delivered electrical stimulation pulse to the VN. In another aspect, the external device 120 may control the pupillometry system 114 to either make or save (or store) pupil measurements at the appropriate times. Some examples of an external device may be a patient remote or clinician controller. The patient remote may be software that is loaded onto a mobile device or computing device of a subject. The clinician controller may be a dedicated device used with the VNS system 100.

In some aspects, synchronization of the pupillometry system 114 and the VNS system 100 may be performed at an internal (e.g., implanted) device corresponding to a wirelessly connected discrete controller, a wire-connected discrete controller, or a controller integrated with or housed within the stimulation system. In some aspects, the synchronization of the pupillometry system 114 and the VNS system 100 may be performed remotely by cloud computing via data communicated wirelessly from either external or internal (e.g., implanted) parts of the stimulation system.

The pupillometry system 114 may then transmit either each individual pupil measurement or a difference between the stimulation on and stimulation off pupil measurements to the external device 120. Depending on the noise and other sources of error, the external device 120 may make multiple pupil differential size measurements to look for and eliminate those sources of error to yield a filtered pupil response measurement. The external device 120 may then make one or more filtered pupil response measurements after simulating the vagus nerve with each electrode while increasing the stimulation pulse amplitude. The external device 120 may determine a minimum stimulation amplitude where recruitment begins for some afferent fibers. In some examples, the external device 120 may also determine a maximum stimulation amplitude. These thresholds may be saved (or stored) and used as a set of minimum stimulation amplitudes in subsequent treatments for the subject. The external device 120 may also use these thresholds to select any number of the electrodes 112 with the greatest response or de-select any number of the electrodes 112 with the worst response from any stimulation programs.

As previously mentioned, although an electrode (cathode, anode, or pair of electrodes) may elicit the greatest pupillary response (and, presumably, greatest therapeutic effect), the same electrode and/or pair of electrodes may also result in undesirable side effects for the subject. In this case, the information regarding undesirable side effects may also be collected and used to modify an algorithm employed to select the indicated electrode or electrode pair. In some aspects, an EMG sensor 116 may be used to detect and report undesired side effects from cervical vagus stimulation. This information may also be used to adjust a therapeutic score assigned to an electrode or electrode pair 112 (e.g., electrode or electrode pair).

In some aspects, the external device 120 may be configured to execute a user application configured to communicate with a clinical application via an intervening cloud infrastructure, allowing a remote clinician to interact with the external controller or the implanted controller. This configuration may allow for a clinician to view sensor measurement data, to view and to modify one or more settings of the VNS system 100, and/or to allow a clinician to view long term data and/or trends after each adjustment or reading to determine if the stimulator efficacy is changing over time. For example, the clinician may be able to edit stimulation profile or individual parameters stored on the external device 120, which may in turn be transmitted to the implanted controller in order to modify the treatment regimen or stimulation parameters applied by the VNS system 100.

The implanted controller and/or the external device 120 may also be configured to communicate with one or more local, remote, or cloud-based servers 130. For example, in this case, the external device 120 may be capable of communicating with a remote server 140 via intermediary cloud-based infrastructure.

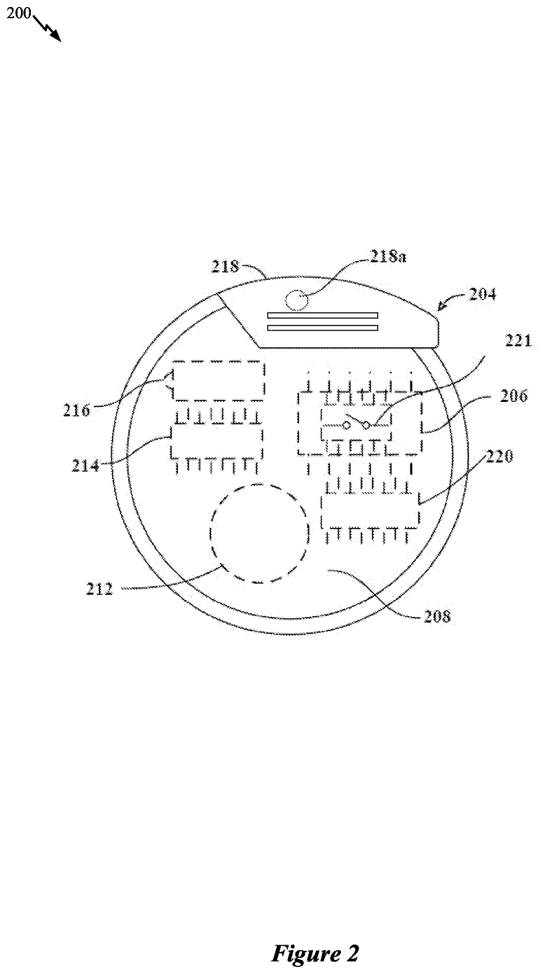

Although FIG. 1A, shows methods and procedures being performed by the external device 120, the role and functionalities of the external device 120 may be performed by a controller (e.g., the controller 220 in FIG. 2) on the VNS stimulator 110 and/or the pupillometry system 114. In some aspects, the external device 120 may be included in any one of a smart phone, a tablet, or any other similar device.

FIG. 1B is a diagram illustrating an exemplary embodiment of a VNS system for treating epilepsy using a VNS stimulator 110 implanted under the skin in the chest of a subject and coupled via a lead wire 104 to drive a cuff electrode 108 (or a plurality of electrodes inside the cuff) on the vagus nerve 102. FIG. 1B is a close-up version of the VNS stimulator 110 and the vagus nerve 102 shown in FIG. 1A.

The VNS stimulator 110 may also include a rechargeable or primary cell, single-use battery (now shown in FIG. 1B) for transmitting electrical pulses using a selectable pulse width and frequency. The VNS stimulator 110 may further include an outlet portion 106 for enabling a connection between the VNS stimulator 110 and lead wire 104, which enables current flow to the vagus nerve 102 via the cuff electrode 108 and electrode(s) 112, and/or a connection between VNS stimulator 110 and a pupillometry system (also not shown in FIG. 1B).

In FIG. 1B, four electrode pairs 112a, 112b, 112c, and 112d are connected to different portions of the vagus nerve 102. However, any number of electrode pairs (e.g., a cathode and an anode) may be used to connect the vagus nerve 102. The electrodes may be selectively activated, e.g., to identify acceptable amplitudes for the different electrodes. In some embodiments, a pair of electrodes are used. In some embodiments, multiple pairs of electrodes are placed at a multiple locations on the vagus nerve.

In addition to determining a minimum threshold assessment for a single electrode, the external device 120 or VNS controller (e.g., the controller 220 in FIG. 2) may also determine the minimum threshold for different pairs of electrodes 112a, 112b, 112c, and 112d. For example, a set of thresholds may be found for each electrode for every pulse width. When an electrode pair is used to deliver stimulation, the stimulation amplitude for afferent fiber recruitment will be lower than the sum of stimulation amplitudes for each individual electrode when used by itself. This is the reason electrode pairs are treated as unique electrodes during the titration process.

As a non-limiting example, five electrodes may be used as a cathode or anode such that a search for stimulation thresholds may be repeated for each potential cathode or anode. In some examples, there may be two modes of operation: axial tripolar, in which two guard bands (proximal and distal of the ring of the electrodes) are used as the return electrode, and radial bipolar, in which one of the cathode's or anode's neighboring electrodes (on the center ring of the electrode) is used as the return electrode.

The VNS system 100 may be used after an initial system implantation and periodically post implantation (e.g., yearly) in order to re-assess the effectiveness of the therapy. Reassessment of minimum stimulation thresholds may be repeated after wound healing occurs post implantation and revision surgery since tissue remodeling will cause impedance changes and dimensional changes between the electrodes that are used and the afferent nerve fibers. These changes will affect the minimum stimulation thresholds. Accordingly, the clinician may want to re-assess the minimum stimulation thresholds on an annual or semi-annual basis in order to see if there have been any changes (e.g., cuff rotation) that may impact the stimulation efficiency. The ability to view long term data and trends on an external device 120 after each adjustment can also help determine if the stimulator efficacy is changing over time or identify patterns.

Pupillometry may also be utilized at an initial fitting and, optionally, at each follow-up visit to confirm no lead motion. Alternatively, pupillometry may be utilized at follow-up visits if a subject's seizure rate has increased and cuff motion is suspected.

FIG. 2 is a diagram illustrating an exemplary embodiment of a VNS stimulator 204 (e.g., the VNS stimulator 110 shown in FIGS. 1A-1B) including a controller 200 with processing circuitry configured to modulate at least one stimulation parameter of the electrical stimulation pulses based at least in part on a measurement of a biological marker during titration. The VNS stimulator 204 may further comprise a pulse generator 206 which is programmed to generate a periodic electrical pulse having a set frequency and pulse width. The pulse generator may be battery-powered and can be activated and deactivated (the latter causing the current to be turned off) via a switch 221. The connections on the integrated circuits may be coupled together selectively via a small, printed circuit board 208. In some embodiments, the VNS stimulator 204 may be implemented as a system on a chip (SoC) on a die, or a packaged die.

The VNS stimulator 204 further may include a transceiver/receiver 216. In some embodiments, transceiver 216 includes a wireless receiver configured to receive wireless signals, e.g., Bluetooth Low Energy, from a source external to the subject (e.g., for communication with an external device 120, local, remote 140, or cloud-based servers 130, pupillometry system 114, or the EMG sensor 116). In some embodiments, the transceiver 216 may further comprise a wireless transmitter, e.g., for providing feedback to a processor used in a clinician programmer device, or to an external sensor. In still other embodiments, the transceiver 216 may include a wire for receiving information from the vagus nerve or another part of the body. The wire may also extend outside the body for temporary connection to an external sensor or other processing device. The wireless receiver in transceiver 216 may further be configured in some embodiments to receive instructions for the controller 220 to modulate one or more parameters of the electrical stimulation pulse (e.g., to increase the stimulation pulse amplitude, as described herein). The wireless transceiver 216 may further be configured to receive information including events recorded or detected by one or more external sensors (e.g., such as the EMG sensor 116). When acting as a transmitter, the transceiver 216 can provide feedback signals to external sources using data generated by controller 220.

In some embodiments, the information received from the wireless receiver/transceiver 216 may be provided to a memory 214. The controller 220 may access the memory 214 to receive and process instructions to modulate (e.g., increase or decrease the pulse amplitude, pulse width, and/or pulse frequency) one or more parameters of the generated electrical stimulation pulses, or to temporarily deactivate the pulse generator 206. In some embodiments, the controller 220 may access information including physical events detected by one or more implanted, external sensors (e.g., the ECG sensor as described herein), and/or the one or more light sources. The processor may evaluate the detected events, and in these embodiments, modulate the pulse amplitude, pulse width, pulse frequency, ramp-on rate, and/or ramp-off rate of the electrical stimulation pulses and/or command the pupillometry system 114 to obtain filtered pupil response measurements after stimulating with each electrode. The pulse generator 206 may generate the electrical stimulation pulses accordingly, which may be provided to an outlet portion 218 (e.g., outlet portion 106 from FIGS. 1A-1B) to which the lead wire (see lead wire 104 from FIGS. 1A-1B) is attached. In some embodiments, the outlet portion 218 may include an aperture 218a for a jack to be inserted to attach the lead wire.

In some aspects, the controller 220 of the VNS stimulator 110 may also be configured to modulate one or more stimulation parameters according to saved thresholds from the memory or to use the saved thresholds to select or deselect any number of electrodes from any stimulation programs. For instance, the controller 220 may be configured to adjust one or more stimulation parameters (e.g., the pulse width and/or frequency) to an increased level in response to the pupillometry system 114 determining no response to the pupils of the subject after delivering a stimulation pulse to the VN using previous stimulation parameters. However, once the pupillometry system 114 determines a response to the pupils of the subject after delivering the stimulation pulse to the VN using the modified stimulation parameters, the controller 220 of the VNS stimulator 110 may recognize the one or more stimulation parameters (e.g., minimum pulse amplitude) for each electrode of a multi-electrode stimulation cuff that causes an action potential in afferent fibers and store the one or more parameters in memory associated with the electrode.

For instance, the controller 220 may set a frequency and pulse width and an amplitude may be adjusted to determine a minimum threshold where afferent Type B fiber recruitment begins and a maximum threshold where voice or cardiac rate changes occur. This process will be repeated for each electrode and any electrode for which there is either no airway opening or for which stimulation causes an airway closing may be disabled. After the fitting session is complete, the VNS system may deliver stimulation from either a “best” electrode or in a round-robin fashion from any of the enabled electrodes.

In some aspects, the controller 220 of the VNS stimulator 204 may synchronize the pupillometry system 114 and the VNS system 100 by communicating with the VNS stimulator 110 to obtain the timing of the stimulation pulse trains. The controller 220 of the VNS stimulator 110 may be configured to control the pupillometry system 114 to be synchronized with the VNS stimulator 110 for obtaining measurements with stimulation on and off. In another aspect, the controller 220 of the VNS stimulator 204 may control the pupillometry system 114 to either make or save pupil measurements at the appropriate times.

Depending on the noise and other sources of error, the controller 220 of the VNS stimulator 204 may determine multiple pupil differential size measurements to look for and eliminate those sources of error to yield a filtered pupil response measurement. The controller 220 may then make one or more filtered pupil response measurements after simulating the vagus nerve with each electrode while increasing the stimulation pulse amplitude. The controller 220 may then determine a minimum stimulation amplitude where recruitment begins for some afferent fibers. These thresholds may be saved and used as a set of minimum stimulation amplitudes in subsequent treatments. The controller 220 may also use these thresholds to select and/or de-select any number of the electrodes with the greatest response or eliminate any number of the electrodes with the worst response from any stimulation programs and/or causing the most undesirable side effects.

As indicated above, the controller 220 of the VNS stimulator 204 may be configured to determine a minimum pulse amplitude for each electrode of a multi-electrode stimulation cuff that causes an action potential in afferent fibers. This allows for quicker titration of stimulation pulse amplitude(s) to levels that cause a therapeutic effect on the subject. In addition, this may allow for automatic titration of stimulation amplitude to the point where seizure occurrence is reduced with minimal stimulator use and minimal current consumption.

While various functions of the VNS stimulator 204 have been shown, it will be appreciated by those skilled in the art upon review of this disclosure that different architectures may be used. For example, the controller 220 may include more than one integrated circuit, or it may include a separate module coupled to the VNS stimulator 204. The controller 220 may further include one or more general purpose processors, reduced instruction set computer (RISC) processors, or other types of processors. The controller 220 may in some embodiments include dedicated hardware. For example, the controller 220 may be any one or more of a digital signal processor (DSP), a field programmable gate array (FPGA), an application-specific integrated circuit (ASIC), or a combination of digital logic devices.

The memory 214 may include any suitable memory, such as a combination of volatile and non-volatile memory, dynamic random access memory (DRAM), static random access memory (SRAM), read only memory, flash or other solid state memory, or the like. Other types of memory are possible. Non-volatile memory within memory 214 may be used to store critical settings to enable system reset, for example. Minimum stimulation parameters (e.g., pulse frequency, pulse amplitude, and/or pulse width values) determined to cause an action potential in afferent fibers may be stored in the memory. In some embodiments, the memory may be accessible and programmable as noted above via the controller 220, or via data or instructions received at via wireless receiver 216. The memory 214 may also include firmware for use by the controller, or other program information for automatically modulating one or more parameters of the electrical pulse in response measurements obtained from the pupillometry system. In some embodiments, the modulation of stimulation parameters may be based, in part or in whole, on information received at the receiver 216 such a detected events. It will be appreciated that VNS 204 does not have to be circular or elliptical in nature, and may take on different shapes based on different design considerations and subject needs. More generally, the components identified in the various figures may take on different geometries than those shown.

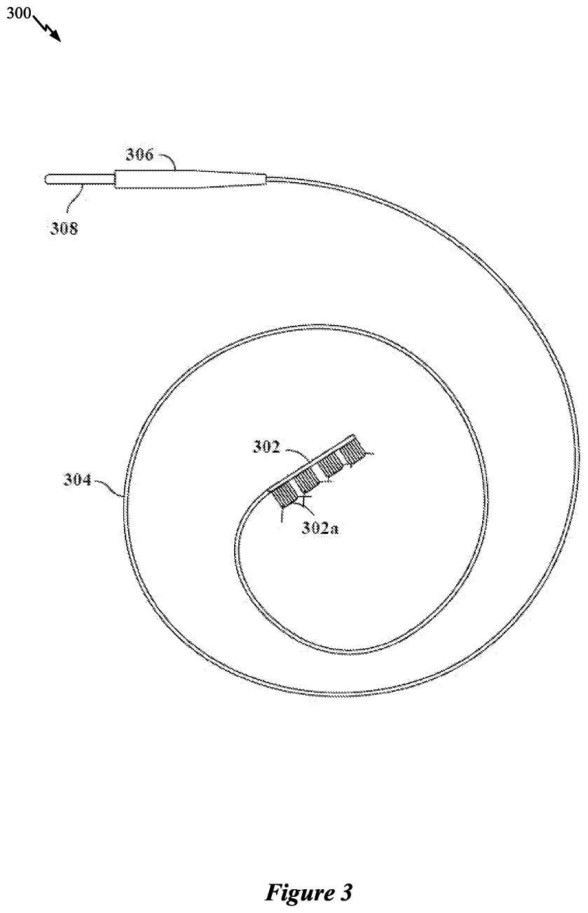

FIG. 3 is a diagram 300 illustrating an exemplary embodiment of a wire and electrode cuff for use in VNS stimulation. The jack 308 may be inserted in one configuration into the aperture 218a (FIG. 2) of VNS stimulator 204. It will be appreciated that the components are not drawn to scale, and typically the jack 308 in this embodiment would be sized very small, and/or angled differently, to be minimally intrusive to the subject in which the component is implanted. In some embodiments, an insulating sheath 306 may provide support for the jack 308 and the insulated wire 304 terminates at electrode cuff or lead 302, which further illustrates a plurality of electrodes 302a or electrode pairs (e.g., electrode pairs 112a, 112b, 112c, and 112d from FIG. 1A). A bipolar nerve cuff electrode has two contacts for current flow — a cathode and an anode. A first contact is a cathode which depolarizes the membrane towards more positive potentials, where the action potential (AP) is generated. A second contact is an anode which injects current and hyperpolarizes the axon membrane potential towards more negative potentials and can arrest AP propagation.

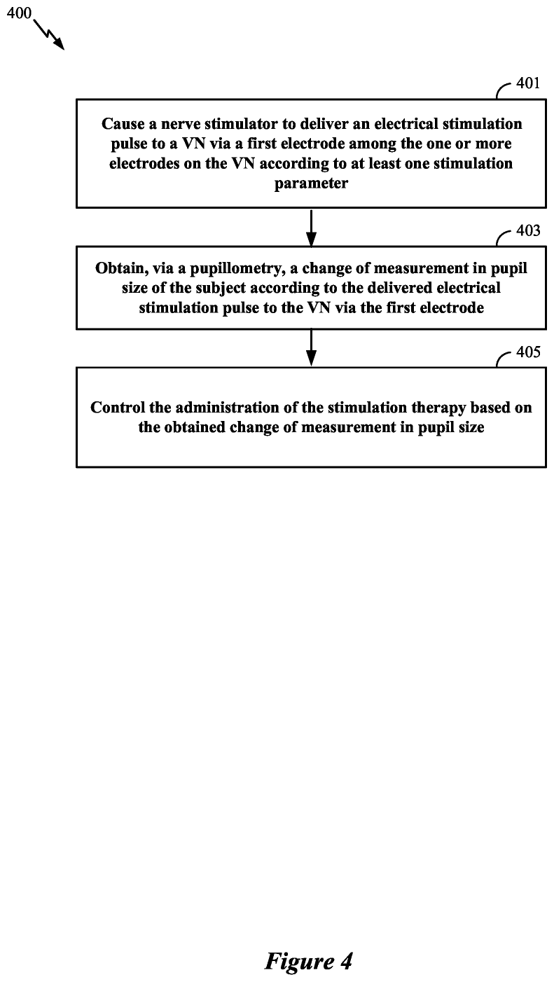

FIG. 4 is a conceptual flowchart summarizing a method for therapy via stimulation in a subject. According to various different aspects, one or more of the illustrated blocks of method 400 may be omitted, transposed, and/or contemporaneously performed. The method 400 allows a system (e.g., the VNS system 100 shown in FIGS. 1A and 1B) to treat epilepsy, depression, or other conditions by synchronizing biological marker measurements with delivery of stimulation to the VN.

The method 400 may be performed by a system, as described above. In some implementations, the method 400 is performed by processing logic, including hardware, firmware, software, or a combination thereof. In some implementations, the method 400 is performed by a processor executing code stored in a non-transitory computer-readable medium (e.g., a memory).

A system performing the method 400 may include a nerve stimulator (VNS stimulator 110 and VNS stimulator 204 shown in FIGS. 1A-2) implanted in the subject and configured to transmit electrical stimulation pulses to a vagus nerve via one or more electrodes (e.g., two or more electrodes and one or more cathodes with at least one anode as a return), and a pupillometry sensor (e.g., the pupillometry system 114 shown in FIGS. 1A-1B) worn by a subject and configured to measure a pupil size of the subject.

At block 401, the method 400 may include causing the nerve stimulator to deliver an electrical stimulation pulse to the VN via a first electrode among the one or more electrodes on the VN according to at least one stimulation parameter. For example, referring back to FIGS. 1A-1B, the VNS stimulator 110 is coupled via a lead wire 104 to drive a cuff electrode 108 (or a plurality of electrodes inside the cuff) on the vagus nerve 102. In some aspects, the at least one stimulation parameter includes at least one of a pulse amplitude or a pulse width, a pulse frequency, ramp-on rate, ramp-off rate, and/or duty cycle of the electrical stimulation pulse. For example, for each electrode, a VN may use one frequency and one pulse width such that an amplitude may be adjusted to find a minimum threshold where afferent Type B fiber recruitment begins and a maximum threshold where voice or cardiac rate changes occur. In some instances, a set of thresholds will be determined for each electrode for every given pulse width.

At block 403, the method 400 may include obtaining, via the pupillometry sensor, a change of measurement in pupil size of the subject according to the delivered electrical stimulation pulse to the VN via the first electrode. These actions will be made based on a change in pupil size from a non-stimulation condition to a stimulation condition. There may be several measurements taken in each state to filter out any impact of light, emotional state, and/or arousal state. For example, referring back to FIG. 1A, the pupillometry system 114 measures a spontaneous variation of the pupil diameter and the pupillary light reflex in a subject.

In some aspects, the obtaining the change of measurement in pupil size of the subject according to the delivered stimulation to the VN further may comprise obtaining, via the pupillometry sensor, a first pupil size measurement when the nerve stimulator delivers an electrical stimulation pulse to the VN via the first electrode among the one or more electrodes according to the at least one stimulation parameter, obtaining, via the pupillometry sensor, a second pupil size measurement when the VNS ceases delivering electrical stimulation pulses to the VN via the first electrode among the one or more electrodes; and storing, in the memory, the first pupil size measurements associated with the one or more parameters and the second pupil size measurement or a delta between the first pupil size measurement and the second pupil size measurement. In some aspects, the first pupil size measurement and the second pupil size measurement of the subject are obtained based on a timing of a stimulation pulse train from the VNS.

At block 405, the method 400 may include controlling administration of the stimulation therapy based on the obtained change of measurement in pupil size. In some aspects, the controlling the administration of the stimulation therapy further comprises administering, ceasing administration, titrating or adjusting a level of electrical stimulation pulse administered to the subject.

In some aspects, the method may further include allowing the subject and/or a remote operator to adjust the at least one stimulation parameter. In some aspects, the controller may allow the user to provide feedback to stimulation comfort level or discomfort. For example, referring back to FIG. 1A, a clinician may adjust at least one stimulation parameter from an external device 120 that communicates with the VNS system 100.

In some aspects, the method 400 may include obtaining a selection of an electrode from among the one or electrodes. In some aspects, the method 400 may include causing the VNS to deliver electrical stimulation pulse to the VN via the selected electrode according to the at least one stimulation parameter.

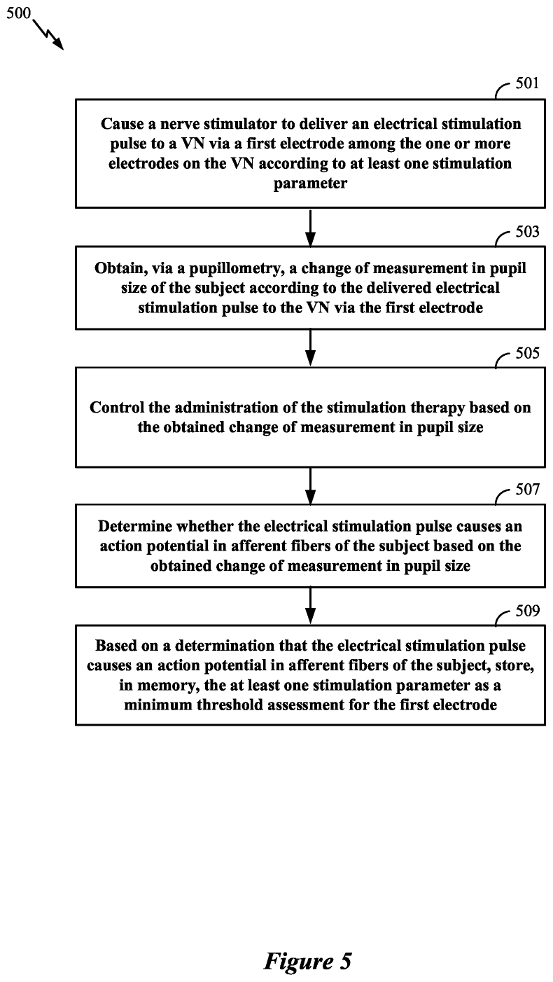

FIG. 5 is a conceptual flowchart summarizing a method for stimulation therapy in a subject. According to various different aspects, one or more of the illustrated blocks of method 500 may be omitted, transposed, and/or contemporaneously performed. The method 500 allows a system (e.g., the VNS system 100 shown in FIGS. 1A-1B) to treat epilepsy, depression, or other conditions by synchronizing biological marker measurements with delivery of stimulation to the VN.

The method 500 may be performed by a system, as described above. In some implementations, the method 500 is performed by processing logic, including hardware, firmware, software, or a combination thereof. In some implementations, the method 500 is performed by a processor executing code stored in a non-transitory computer-readable medium (e.g., a memory).

A system performing the method 500 may include a nerve stimulator (VNS stimulator 110 and VNS stimulator 204 shown in FIGS. 1-2) implanted in the subject and configured to transmit electrical stimulation pulses to a VN of a subject via one or more electrodes, a pupillometry sensor (e.g., the pupillometry system 114 shown in FIG. 1A) worn by a subject and configured to measure a pupil size of the subject, and a EMG sensor configured to obtain EMG measurements of the subject.

At block 501, the method 400 may include causing the nerve stimulator to deliver an electrical stimulation pulse to the VN via a first electrode among the one or more electrodes on the VN according to at least one stimulation parameter. For example, referring back to FIGS. 1A-1B, the VNS stimulator 110 is coupled via a lead wire 104 to drive a cuff electrode 108 (or a plurality of electrodes inside the cuff) on the vagus nerve 102.

At block 503, the method 500 may include obtaining, via the pupillometry sensor, a change of measurement in pupil size of the subject according to the delivered electrical stimulation pulse to the VN via the first electrode. For example, referring back to FIG. 1A, the pupillometry system 114 measures a spontaneous variation of the pupil diameter and the pupillary light reflex in a subject.

At block 505, the method 500 may include controlling administration of the stimulation therapy based on the obtained change of measurement in pupil size. In some aspects, the controller is further configured to allow the subject and/or a remote operator to adjust the at least one stimulation parameter. For example, referring back to FIG. 1A, a clinician may adjust at least one stimulation parameter from an external device 120 that communicates with the VNS system 100.

At block 507, the method 500 may include determining whether the electrical stimulation pulse causes an action potential in afferent fibers of the subject based on the obtained change of measurement in pupil size. For instance, the VNS system may determine an activation of Type B (mid-sized) afferent fibers within the VN. In some aspects, the determining is performed at an external device corresponding to a discrete controller of the stimulation system, a computer, a smart phone, or a tablet. For example, referring back to FIG. 1A, an external device 120 that communicates with the VNS system 100 may perform the determination step.

At block 509, the method 500 may include based on a determination that the electrical stimulation pulse causes an action potential in afferent fibers of the subject, store, in the memory, the at least one stimulation parameter as a minimum threshold parameter for the first electrode.

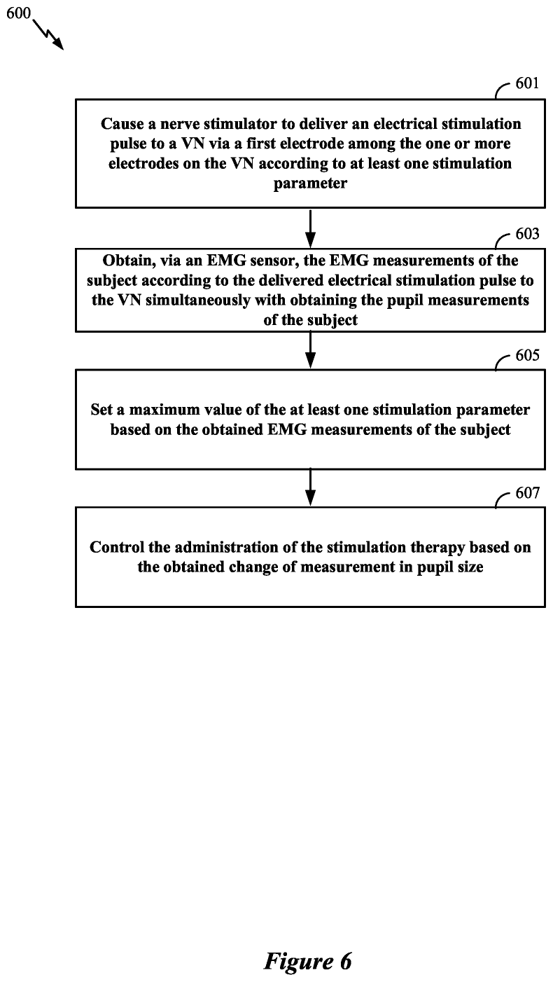

FIG. 6 is a conceptual flowchart summarizing a method for stimulation therapy in a subject. According to various different aspects, one or more of the illustrated blocks of method 600 may be omitted, transposed, and/or contemporaneously performed. The method 600 allows a system (e.g., the VNS system 100 shown in FIGS. 1A-1B) to treat epilepsy by synchronizing biological marker measurements with delivery of stimulation to the VN.

The method 600 may be performed by a system, as described above. In some implementations, the method 600 is performed by processing logic, including hardware, firmware, software, or a combination thereof. In some implementations, the method 600 is performed by a processor executing code stored in a non-transitory computer-readable medium (e.g., a memory).

A system performing the method 600 may include a vague nerve stimulator (VNS stimulator 110 and VNS stimulator 204 shown in FIGS. 1-2) implanted in the subject and configured to transmit electrical stimulation pulses to a VN of a subject via one or more electrodes, a pupillometry sensor (e.g., the pupillometry system 114 shown in FIG. 1A) worn by a subject and configured to measure a pupil size of the subject, and a EMG sensor configured to obtain EMG measurements of the subject.

At block 601, the method 600 may include causing the nerve stimulator to deliver an electrical stimulation pulse to the VN via a first electrode among the one or more electrodes on the VN according to at least one stimulation parameter. For example, referring back to FIGS. 1A-1B, the VNS stimulator 110 is coupled via a lead wire 104 to drive a cuff electrode 108 (or a plurality of electrodes inside the cuff) on the vagus nerve 102.

At block 603, the method 600 may include obtaining, via the EMG sensor, the EMG measurements of the subject according to the delivered electrical stimulation pulse to the VN simultaneously with obtaining the pupil measurements of the subject. For instance, when an EMG signal is detected, the VN is stimulating the VN at an amplitude and pulse width that is too great (i.e., too much charge per phase and/or stimulation pulse).

As an example, an electrode eliciting a greatest response may also result in deleterious side effects in the subject. In these cases, the EMG sensor may collect information and use this information to modify the algorithms for selecting an electrode (cathode or anode) or electrode pair in subsequent treatments. Specifically, the EMG sensor may detect and report the deleterious side effects from cervical vagus stimulation and use this information to adjust a therapeutic score assigned to an electrode or electrode pair. For example, referring back to FIG. 1A, the EMG sensor 116 may obtain EMG measurements from the subject.

At block 605, the method 600 may include setting a maximum value of the at least one stimulation parameter based on the obtained EMG measurements of the subject. In some examples, the method 600 may also include setting a minimum value of the at least one stimulation parameter based on the obtained EMG measurements. In some examples, both may be measured and used to set minimum and maximum thresholds and then the pulse amplitude may be titrated from the minimum threshold to the maximum threshold over several weeks. Upon a subject's subsequent follow-up visit, a remote operator of clinician may re-assess the maximum value.

At block 607, the method 600 may include controlling administration of the stimulation therapy based on the obtained change of measurement in pupil size.

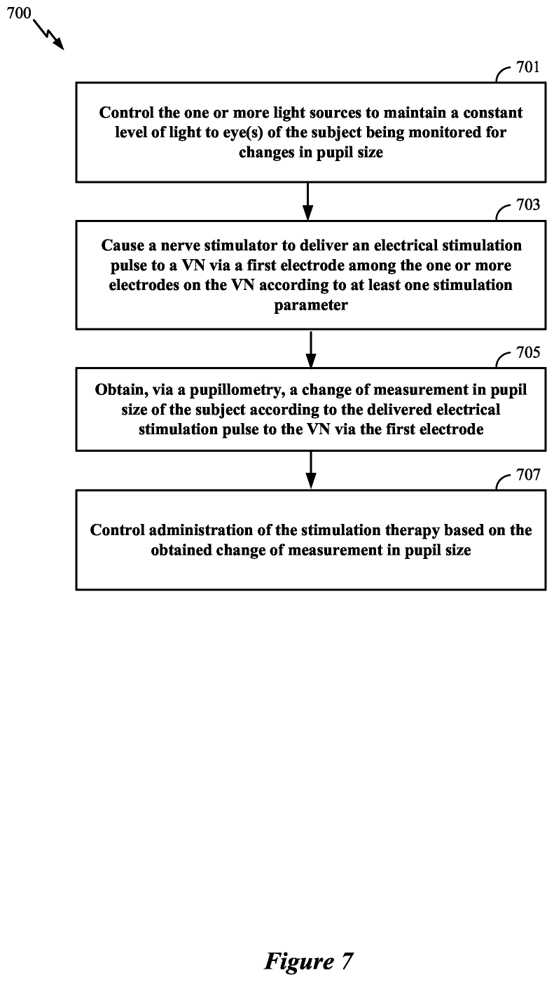

FIG. 7 is a conceptual flowchart summarizing a method for stimulation therapy in a subject. According to various different aspects, one or more of the illustrated blocks of method 700 may be omitted, transposed, and/or contemporaneously performed. The method 700 allows a system (e.g., the VNS system 100 shown in FIGS. 1A-1B) to treat epilepsy, depression, or other conditions by synchronizing biological marker measurements with delivery of stimulation to the VN.

The method 700 may be performed by a system, as described above. In some implementations, the method 700 is performed by processing logic, including hardware, firmware, software, or a combination thereof. In some implementations, the method 700 is performed by a processor executing code stored in a non-transitory computer-readable medium (e.g., a memory).

A system performing the method 700 may include a nerve stimulator (VNS stimulator 110 and VNS stimulator 204 shown in FIGS. 1-2) implanted in the subject and configured to transmit electrical stimulation pulses to a VN of a subject via one or more electrodes, a pupillometry sensor (e.g., the pupillometry system 114 shown in FIG. 1A) worn by a subject and configured to measure a pupil size of the subject, and one or more light sources coupled to the pupillometry sensor.

At block 701, the method 700 may include controlling the one or more light sources to obtain a nominal pupil size of the subject. In some aspects, a light source (e.g., LED) is added into a side of pupillometry system to control the ambient lighting of the pupil and the pupil's response to ambient light. In some aspects, the method 700 may further include controlling the one or more light sources to maintain a constant level of light to eye(s) of the subject being monitored for changes in pupil size.

At block 703, the method 700 may include causing the nerve stimulator to deliver an electrical stimulation pulse to the VN via a first electrode among the one or more electrodes on the VN according to at least one stimulation parameter. For example, referring back to FIGS. 1A-1B, the VNS stimulator 110 is coupled via a lead wire 104 to drive a cuff electrode 108 (or a plurality of electrodes inside the cuff) on the vagus nerve 102.

At block 705, the method 700 may include obtaining, via the pupillometry sensor, a change of measurement in pupil size of the subject according to the delivered electrical stimulation pulse to the VN via the first electrode. For example, referring back to FIG. 1A, the pupillometry system 114 measures a spontaneous variation of the pupil diameter and the pupillary light reflex in a subject.

At block 707, the method 700 may include controlling administration of the stimulation therapy based on the obtained change of measurement in pupil size. In some aspects, the controlling the administration of the stimulation therapy further comprises administering, ceasing administration, titrating or adjusting a level of electrical stimulation pulse administered to the subject.

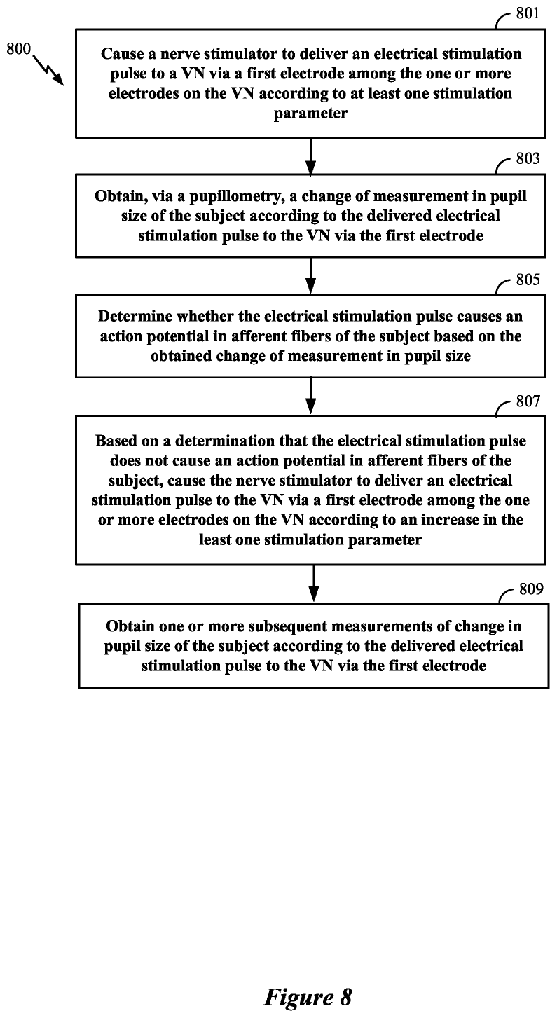

FIG. 8 is a conceptual flowchart summarizing a method for stimulation therapy in a subject. According to various different aspects, one or more of the illustrated blocks of method 600 may be omitted, transposed, and/or contemporaneously performed. The method 800 allows a system (e.g., the VNS system 100 shown in FIGS. 1A-1B) to treat epilepsy, depression, or other conditions by synchronizing biological marker measurements with delivery of stimulation to the VN.

The method 800 may be performed by a system, as described above. In some implementations, the method 800 is performed by processing logic, including hardware, firmware, software, or a combination thereof. In some implementations, the method 800 is performed by a processor executing code stored in a non-transitory computer-readable medium (e.g., a memory).

A system performing the method 800 may include a nerve stimulator (VNS stimulator 110 and VNS stimulator 204 shown in FIGS. 1-2) implanted in the subject and configured to transmit electrical stimulation pulses to a VN of a subject via one or more electrodes, a pupillometry sensor (e.g., the pupillometry system 114 shown in FIG. 1A) worn by a subject and configured to measure a pupil size of the subject, and one or more light sources coupled to the pupillometry sensor.

At block 801, the method 800 may include causing the nerve stimulator to deliver an electrical stimulation pulse to the VN via a first electrode among the one or more electrodes on the VN according to at least one stimulation parameter. For example, referring back to FIGS. 1A-1B, the VNS stimulator 110 is coupled via a lead wire 104 to drive a cuff electrode 108 (or a plurality of electrodes inside the cuff) on the vagus nerve 102.

At block 803, the method 800 may include obtaining, via the pupillometry sensor, a change of measurement in pupil size of the subject according to the delivered electrical stimulation pulse to the VN via the first electrode. For example, referring back to FIG. 1A, the pupillometry system 114 measures a spontaneous variation of the pupil diameter and the pupillary light reflex in a subject.

At block 805, the method 800 may include determining whether the electrical stimulation pulse causes an action potential in afferent fibers of the subject based on the obtained change of measurement in pupil size. This response indicates that an activation potential for at least one Type B afferent fiber has been reached based on a magnitude of the pupil response.

In some aspects, the determining is performed at an external device corresponding to a discrete controller of the stimulation system, a computer, a smart phone, or a tablet. For example, referring back to FIG. 1A, an external device 120 that communicates with the VNS system 100 may perform the determination step.

At block 807, the method may include based on a determination that the electrical stimulation pulse does not cause an action potential in afferent fibers of the subject, causing the nerve stimulator to deliver an electrical stimulation pulse to the VN via a first electrode among the one or more electrodes on the VN according to an increase in the least one stimulation parameter. This is because the previous stimulation parameters may not have caused enough of an electrical stimulation pulse to cause an action potential in afferent fibers. Accordingly, the VNS system may increase a stimulation parameter in a subsequent stimulation.

At block 809, the method may include obtaining one or more subsequent measurements of change in pupil size of the subject according to the delivered electrical stimulation pulse to the VN via the first electrode.

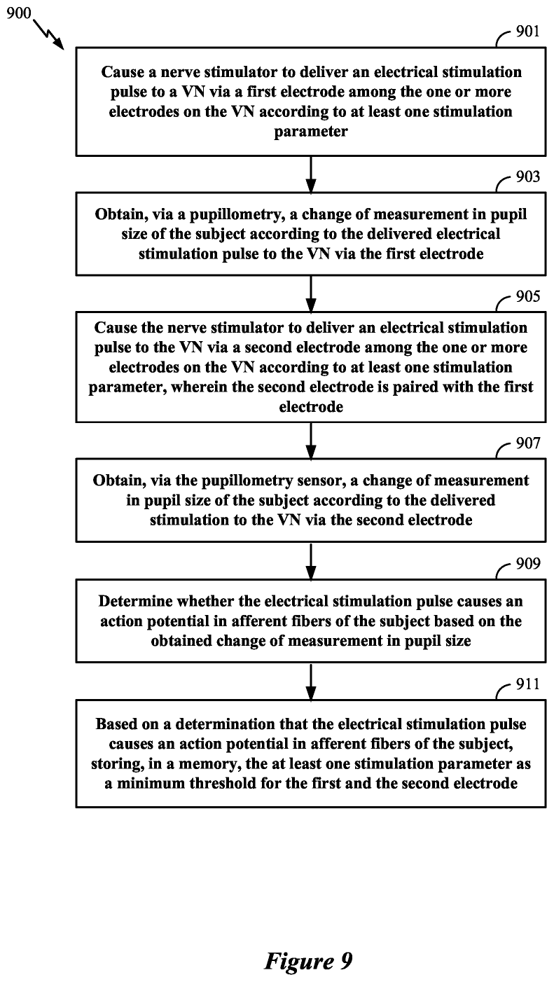

FIG. 9 is a conceptual flowchart summarizing a method for stimulation therapy in a subject. According to various different aspects, one or more of the illustrated blocks of method 900 may be omitted, transposed, and/or contemporaneously performed. The method 900 allows a system (e.g., the VNS system 100 shown in FIGS. 1A-1B) to treat epilepsy, depression, or other conditions by synchronizing biological marker measurements with delivery of stimulation to the VN.

The method 900 may be performed by a system, as described above. In some implementations, the method 900 is performed by processing logic, including hardware, firmware, software, or a combination thereof. In some implementations, the method 900 is performed by a processor executing code stored in a non-transitory computer-readable medium (e.g., a memory).

A system performing the method 900 may include a nerve stimulator (VNS stimulator 110 and VNS stimulator 204 shown in FIGS. 1-2) implanted in the subject and configured to transmit electrical stimulation pulses to a VN of a subject via one or more electrodes, a pupillometry sensor (e.g., the pupillometry system 114 shown in FIG. 1A) worn by a subject and configured to measure a pupil size of the subject, and one or more light sources coupled to the pupillometry sensor.

At block 901, the method 900 may include causing the nerve stimulator to deliver an electrical stimulation pulse to the VN via a first electrode among the one or more electrodes on the VN according to at least one stimulation parameter. For example, referring back to FIGS. 1A-1B, the VNS stimulator 110 is coupled via a lead wire 104 to drive a cuff electrode 108 (or a plurality of electrodes inside the cuff) on the vagus nerve 102.

At block 903, the method 900 may include obtaining, via the pupillometry sensor, a change of measurement in pupil size of the subject according to the delivered electrical stimulation pulse to the VN via the first electrode. For example, referring back to FIG. 1A, the pupillometry system 114 measures a spontaneous variation of the pupil diameter and the pupillary light reflex in a subject.

At block 905, the method 900 may include causing the nerve stimulator to deliver an electrical stimulation pulse to the VN via a second electrode among the one or more electrodes on the VN according to at least one stimulation parameter, wherein the second electrode is paired with the first electrode. For example, referring back to FIG. 1B, the VNS stimulator 110 is coupled via a lead wire 104 to drive a cuff electrode 108 (or a plurality of electrodes inside the cuff) with pairs of electrodes 112a connected to the vagus nerve.

At block 907, the method 900 may include obtaining, via the pupillometry sensor, a change of measurement in pupil size of the subject according to the delivered stimulation to the VN via the second electrode. For example, referring back to FIG. 1A, the pupillometry sensor from the pupillometry system 114 may obtain pupil size measurements by synching up with the VNS stimulator 110.

At block 909, the method 900 may include determining whether the electrical stimulation pulse causes an action potential in afferent fibers of the subject based on the obtained change of measurement in pupil size. In some aspects, the determining is performed at an external device corresponding to a discrete controller of the stimulation system, a computer, a smart phone, or a tablet. For example, referring back to FIG. 1A, an external device 120 that communicates with the VNS system 100 may perform the determination step.

At block 911, the method 900 may include based on a determination that the electrical stimulation pulse causes an action potential in afferent fibers of the subject, storing, in a memory, the at least one stimulation parameter as a minimum threshold for the first and the second electrode.

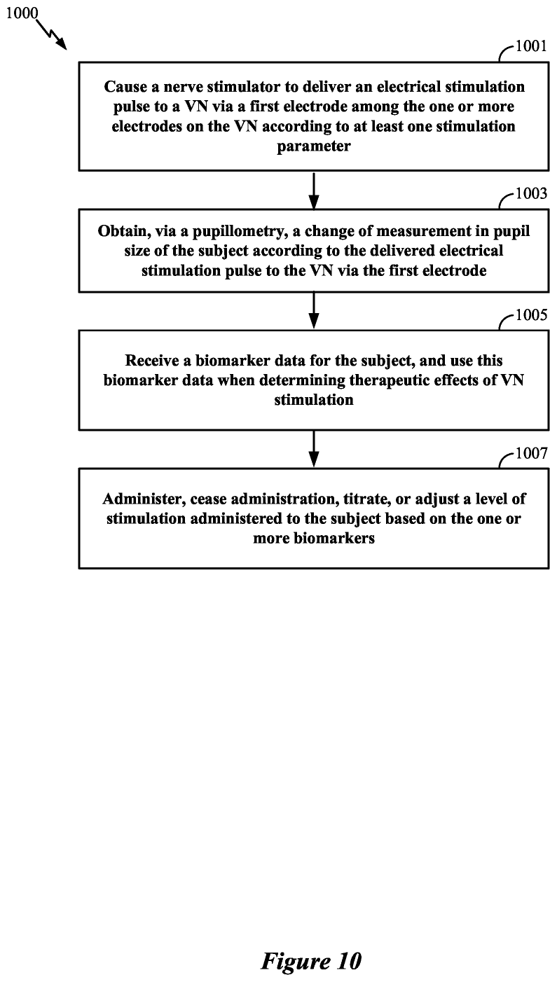

FIG. 10 is a conceptual flowchart summarizing a method for stimulation therapy in a subject. According to various different aspects, one or more of the illustrated blocks of method 1000 may be omitted, transposed, and/or contemporaneously performed. The method 1000 allows a system (e.g., the VNS system 100 shown in FIGS. 1A-1B) to treat epilepsy, depression, or other conditions by synchronizing biological marker measurements with delivery of stimulation to the VN.

The method 1000 may be performed by a system, as described above. In some implementations, the method 1000 is performed by processing logic, including hardware, firmware, software, or a combination thereof. In some implementations, the method 1000 is performed by a processor executing code stored in a non-transitory computer-readable medium (e.g., a memory).

A system performing the method 1000 may include a nerve stimulator (VNS stimulator 110 and VNS stimulator 204 shown in FIGS. 1-2) implanted in the subject and configured to transmit electrical stimulation pulses to a VN of a subject via one or more electrodes, a pupillometry sensor (e.g., the pupillometry system 114 shown in FIG. 1A) worn by a subject and configured to measure a pupil size of the subject, and a sensor configured to detect, measure, and/or monitor one or more biomarkers of the subject.

At block 1001, the method 1000 may include causing the nerve stimulator to deliver an electrical stimulation pulse to the VN via a first electrode among the one or more electrodes on the VN according to at least one stimulation parameter. For example, referring back to FIGS. 1A-1B, the VNS stimulator 110 is coupled via a lead wire 104 to drive a cuff electrode 108 (or a plurality of electrodes inside the cuff) on the vagus nerve 102.

At block 1003, the method 1000 may include obtaining, via the pupillometry sensor, a change of measurement in pupil size of the subject according to the delivered electrical stimulation pulse to the VN via the first electrode. For example, referring back to FIG. 1A, the pupillometry system 114 measures a spontaneous variation of the pupil diameter and the pupillary light reflex in a subject.

At block 1005, the method 1000 may include receiving a biomarker data for the subject, and to use this biomarker data when determining therapeutic effects of VN stimulation. In some examples, the biomarker comprises a heart rate variability (HVR) of the subject.

At block 1007, the method 1000 may include administering, ceasing administration, titrate, or adjusting a level of stimulation administered to the subject based on the one or more biomarkers.

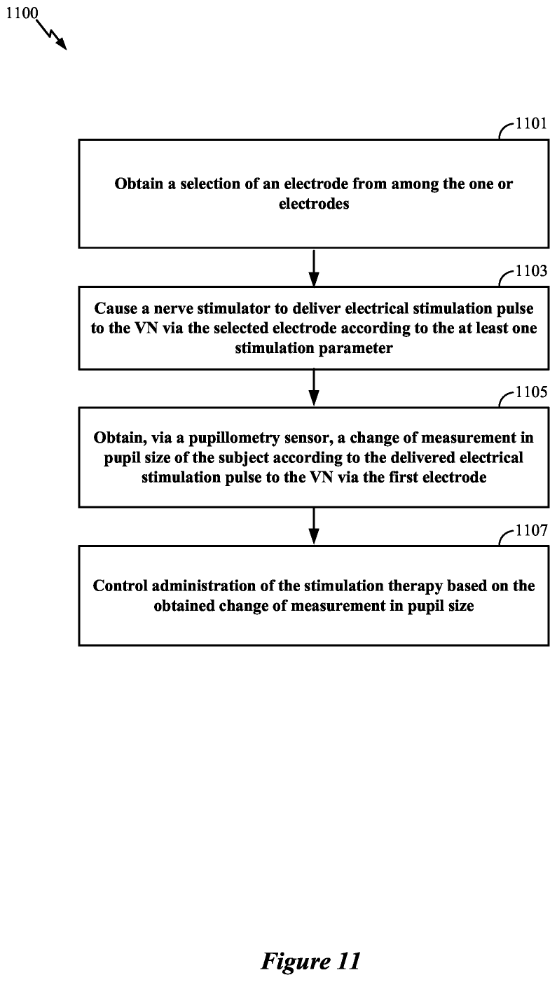

FIG. 11 is a conceptual flowchart summarizing a method for stimulation therapy in a subject. According to various different aspects, one or more of the illustrated blocks of method 1100 may be omitted, transposed, and/or contemporaneously performed. The method 1100 allows a system (e.g., the VNS system 100 shown in FIGS. 1A-1B) to treat epilepsy, depression, or other conditions by synchronizing biological marker measurements with delivery of stimulation to the VN.

The method 1100 may be performed by a system, as described above. In some implementations, the method 1100 is performed by processing logic, including hardware, firmware, software, or a combination thereof. In some implementations, the method 1100 is performed by a processor executing code stored in a non-transitory computer-readable medium (e.g., a memory).

A system performing the method 1100 may include a nerve stimulator (VNS stimulator 110 and VNS stimulator 204 shown in FIGS. 1-2) implanted in the subject and configured to transmit electrical stimulation pulses to a VN of a subject via one or more electrodes, and a pupillometry sensor (e.g., the pupillometry system 114 shown in FIG. 1A) worn by a subject.

At block 1101, the method 1100 may include receiving a selection of an electrode from among the one or electrodes. As shown in FIG. 1B, the electrode may be any of the four electrode pairs 112a, 112b, 112c, and 112d connected to different portions of the vagus nerve. The electrodes may be selectively activated, e.g., to identify acceptable amplitudes for the different electrodes. As a non-limiting example, five electrodes may be used as an electrode such that a search for stimulation thresholds may be repeated for each potential electrode. In some examples, there may be two modes of operation: axial tripolar, in which two guard bands (proximal and distal of the ring of the electrodes) are used as the return electrode, and radial bipolar, in which one of the cathode's neighboring electrodes (on the center ring of the electrode) is used as the return electrode.

At block 1103, the method 1100 may include causing the nerve stimulator to deliver electrical stimulation pulse to the VN via the selected electrode according to the at least one stimulation parameter. For example, referring back to FIGS. 1A-1B, the VNS stimulator 110 is coupled via a lead wire 104 to drive a selected electrode from the cuff electrode 108 (or a plurality of electrodes inside the cuff) on the vagus nerve 102.

At block 1105, the method 1100 may include obtaining, via the pupillometry sensor, a change of measurement in pupil size of the subject according to the delivered electrical stimulation pulse to the VN via the first electrode. For example, referring back to FIG. 1A, the pupillometry system 114 measures a spontaneous variation of the pupil diameter and the pupillary light reflex in a subject.

At block 1107, the method 1100 may include controlling administration of the stimulation therapy based on the obtained change of measurement in pupil size.

It is understood that the method illustrated by FIGS. 4-11 are exemplary in nature and that the steps described herein may be combined to generate alternative embodiments.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A stimulation system, comprising:
a nerve stimulator implanted in a subject and configured to deliver a stimulation therapy to the subject based on transmitting electrical stimulation pulses to a vagus nerve (VN) of the subject via one or more electrodes; and
a pupillometry sensor associated with the subject and configured to measure a pupil size of the subject, wherein the nerve stimulator comprises a controller communicatively coupled to the nerve stimulator and the pupillometry sensor, the controller configured to:
cause the nerve stimulator to deliver an electrical stimulation pulse to the VN via a first electrode among the one or more electrodes on the VN according to at least one stimulation parameter;
obtain, via the pupillometry sensor, a change of measurement in pupil size of the subject according to the delivered electrical stimulation pulse to the VN via the first electrode; and
control administration of the stimulation therapy based on the obtained change of measurement in pupil size.

2. The stimulation system of claim 1, wherein the controller is further configured to:
determine whether the electrical stimulation pulse causes an action potential in afferent fibers of the subject based on the obtained change of measurement in pupil size; and
based on a determination that the electrical stimulation pulse causes an action potential in afferent fibers of the subject, store, in memory, the at least one stimulation parameter as a minimum threshold parameter for the first electrode.

3. The stimulation system of claim 2, wherein the determining is performed at an external device corresponding to a discrete controller of the stimulation system, a computer, a smart phone, or a tablet.

4. The stimulation system of claim 2, wherein the determining is performed at an internal device corresponding to a wirelessly connected discrete controller, a wire-connected discrete controller, or a controller integrated or housed with the stimulation system. The stimulation system of claim 2, wherein the determining is performed remotely by cloud computing via data communicated wirelessly from either external or internal parts of the stimulation system.

6. The stimulation system of claim 1, wherein the at least one stimulation parameter includes at least one of a pulse amplitude, a pulse width, a pulse frequency, ramp-on rate, ramp-off rate, and/or duty cycle of the electrical stimulation pulses.

7. The stimulation system of claim 1, wherein the stimulation system further comprises:
an electromyography (EMG) sensor configured to obtain EMG measurements of the subject, wherein the controller is further configured to:
obtain, via the EMG sensor, the EMG measurements of the subject according to the delivered electrical stimulation pulse to the VN simultaneously with obtaining the pupil measurements of the subject; and
setting a maximum value of the at least one stimulation parameter based on the obtained EMG measurements of the subject.

8. The stimulation system of claim 1, wherein the stimulation system further comprises:
one or more light sources coupled to the pupillometry sensor, wherein the controller is further configured to:
control the one or more light sources to obtain a nominal pupil size of the subject.

9. The stimulation system of claim 1, wherein the stimulation system further comprises:
one or more light sources coupled to the pupillometry sensor, wherein the controller is further configured to:
control the one or more light sources to maintain a constant level of light to eye(s) of the subject being monitored for changes in pupil size.

10. The stimulation system of claim 1, wherein the obtaining the change of measurement in pupil size of the subject according to the delivered stimulation to the VN further comprises:
obtaining, via the pupillometry sensor, a first pupil size measurement when the nerve stimulator delivers an electrical stimulation pulse to the VN via the first electrode among the one or more electrodes according to the at least one stimulation parameter;
obtaining, via the pupillometry sensor, a second pupil size measurement when the nerve stimulator ceases delivering electrical stimulation pulses to the VN via the first electrode among the one or more electrodes; and
storing, in a memory, one or more parameters associated with, or affected by, the first and/or second pupil size measurements or a delta between the first pupil size measurement and the second pupil size measurement.

11. The stimulation system of claim 10, wherein the first pupil size measurement and the second pupil size measurement of the subject are obtained in relation to a timing of a stimulation pulse train delivered by the nerve stimulator to the VN.

12. The stimulation system of claim 1, wherein the controller is further configured to:
determine whether the electrical stimulation pulse causes an action potential in afferent fibers of the subject based on the obtained change of measurement in pupil size;
based on a determination that the electrical stimulation pulse does not cause an action potential in afferent fibers of the subject, cause the nerve stimulator to deliver an electrical stimulation pulse to the VN via a first electrode among the one or more electrodes on the VN according to an increase in the least one stimulation parameter; and
obtain one or more subsequent measurements of change in pupil size of the subject according to the delivered electrical stimulation pulse to the VN via the first electrode.

13. The stimulation system of claim 1, wherein the controller is further configured to:
cause the nerve stimulator to deliver an electrical stimulation pulse to the VN via a second electrode among the one or more electrodes on the VN according to at least one stimulation parameter, wherein the second electrode is paired with the first electrode;
obtain, via the pupillometry sensor, a change of measurement in pupil size of the subject according to the delivered stimulation to the VN via the second electrode;
determine whether the electrical stimulation pulse causes an action potential in afferent fibers of the subject based on the obtained change of measurement in pupil size; and
based on a determination that the electrical stimulation pulse causes an action potential in afferent fibers of the subject, storing, in memory, the at least one stimulation parameter as a minimum threshold for the first and the second electrode.

14. The stimulation system of claim 1, wherein the stimulation system further comprises:
a sensor configured to detect, measure, and/or monitor one or more biomarkers of the subject, wherein the controller is further configured to:
receive a biomarker data for the subject, and use the biomarker data when determining therapeutic effects of VN stimulation. The stimulation system of claim 14, wherein the one or more biomarker comprises a heart rate variability (HVR) of the subject.

16. The stimulation system of claim 14, wherein the controller is further configured to:
administer, cease administering, titrate, or adjust a level, pulse width, or frequency of stimulation administered to the subject based on the one or more biomarkers.

17. The stimulation system of claim 1, wherein the controller is further configured to:
obtain a selection of an electrode from among the one or electrodes; and
cause the nerve stimulator to deliver electrical stimulation pulse to the VN via the selected electrode according to the at least one stimulation parameter.

18. The stimulation system of claim 1, wherein controlling the administration of the stimulation therapy further comprises administering, ceasing administration, titrating or adjusting a level of electrical stimulation pulse administered to the subject.

19. A method for stimulation, comprising:
providing a nerve stimulator implanted in a subject and configured to deliver a stimulation therapy to a subject based on transmitting electrical stimulation pulses to a vagus nerve (VN) of the subject via one or more electrodes;
providing a pupillometry sensor associated with a subject and configured to measure a pupil size of the subject;
delivering an electrical stimulation pulse to the VN via a first electrode among the one or more electrodes on the VN according to at least one stimulation parameter;
obtaining, via the pupillometry sensor, a change of measurement in pupil size of the subject according to the delivered electrical stimulation pulse to the VN via the first electrode; and
controlling administration of the stimulation therapy based on the obtained change of measurement in pupil size.

20. The method of claim 19, further comprising:
determining whether the electrical stimulation pulse causes an action potential in afferent fibers of the subject based on the obtained change of measurement in pupil size; and
based on a determination that the electrical stimulation pulse causes an action potential in afferent fibers of the subject, storing the at least one stimulation parameter as a minimum threshold for the first electrode.

21. The method of claim 20, wherein the determining is performed at an external device corresponding to a controller, a computer, a smart phone, or a tablet.

22. The method of claim 21, wherein the determining is performed at an internal(implanted) device corresponding to a wirelessly connected discrete controller, wire-connected discrete controller, or controller integrated or housed with a stimulation system.

23. The method of claim 21, wherein the determining is performed remotely by cloud computing via data communicated wirelessly from either external or internal(implanted) parts of a stimulation system.

24. The method of claim 19, wherein the at least one stimulation parameter includes at least one of a pulse amplitude, or a pulse width, a pulse frequency, ramp-on rate, /or ramp-off rate, and/or duty cycle of the electrical stimulation pulse.

25. The method of claim 19, further comprising:
providing an electromyography (EMG) sensor configured to obtain EMG measurements of the subject;
obtaining, via the EMG sensor, the EMG measurements of the subject according to the delivered electrical stimulation pulse to the VN simultaneously with obtaining the pupil measurements of the subject; and
setting a maximum value of the at least one stimulation parameter based on the obtained EMG measurements of the subject.

26. The method of claim 19, further comprising:
providing one or more light sources coupled to the pupillometry sensor; and
controlling the one or more light sources to obtain a nominal pupil size of the subject.

27. The method of claim 19, wherein the obtaining the change of measurement in pupil size of the subject according to the delivered stimulation to the VN further comprises:
obtaining, via the pupillometry sensor, a first pupil size measurement when the nerve stimulator delivers an electrical stimulation pulse to the VN via the first electrode among the one or more electrodes according to the at least one stimulation parameter;
obtaining, via the pupillometry sensor, a second pupil size measurement when the nerve stimulator ceases delivering electrical stimulation pulses to the VN via the first electrode among the one or more electrodes; and
storing, in memory, one or more parameters associated with, or affected by, the first and/or second pupil size measurements or a delta between the first pupil size measurement and the second pupil size measurement.

28. The method of claim 27, wherein the first pupil size measurement and the second pupil size measurement of the subject are obtained based on a timing of a stimulation pulse train at the nerve stimulator.

29. The method of claim 19, further comprising:
determining whether the electrical stimulation pulse causes an action potential in afferent fibers of the subject based on the obtained change of measurement in pupil size;
based on a determination that the electrical stimulation pulse does not cause an action potential in afferent fibers of the subject, causing the nerve stimulator to deliver an electrical stimulation pulse to the VN via a first electrode among the one or more electrodes on the VN according to an increase in the least one stimulation parameter; and
obtaining one or more subsequent measurements of change in pupil size of the subject according to the delivered electrical stimulation pulse to the VN via the first electrode.

30. The method of claim 19, further comprising:
delivering an electrical stimulation pulse to the VN via a second electrode among the one or more electrodes on the VN according to at least one stimulation parameter, wherein the second electrode is paired with the first electrode;
obtaining, via the pupillometry sensor, measurement of change in pupil size of the subject according to the delivered stimulation to the VN via the second electrode;
determining whether the electrical stimulation pulse causes an action potential in afferent fibers of the subject based on the obtained measurement of change in pupil size; and
based on a determination that the electrical stimulation pulse causes an action potential in afferent fibers of the subject, storing the at least one stimulation parameter as a minimum threshold for the first and the second electrode.

31. The method of claim 19, further comprising:
providing a sensor configured to detect, measure, and/or monitor one or more biomarkers of the subject; and
receiving a biomarker data for the subject comprising a concentration or an amount of biomarker of the subject, and to use this biomarker data when determining therapeutic effects of VN stimulation.

32. The method of claim 31, wherein the one or more biomarker comprises at least a heart rate variability (HVR) of the subject.

33. The method of claim 32, further comprising:
administrating, ceasing administration, titrating, or adjusting a level of stimulation administered to the subject based on the one or more biomarkers.

34. The method of claim 19, further comprising:
receiving a selection of an electrode from among the one or electrodes; and
delivering electrical stimulation pulse to the VN via the selected electrode according to the at least one stimulation parameter.

35. The method of claim 19, further comprising:
allowing the subject and/or a remote operator to adjust the at least one stimulation parameter.
